(19) United States
(12) Patent Application Publication
(10) Pub. No.: US 2003/0017219 A1
(43) Pub. Date: Jan. 23, 2003

(54) Carbohydrate modifying agent and drinks containing the modifying agent

(76) Inventors: Frank Corsini, Tiburon, CA (US); Richard L. Kozlenko, San Rafael, CA (US); Joe A. Vinson, (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/155,865

(22) Filed: May 24, 2002

Related U.S. Application Data
(60) Provisional application No. 60/293,657, filed on May 25, 2001.

(51) Int. Cl. A61K 35/78

(52) U.S. Cl. 424/729

(57) ABSTRACT

The present invention provides a composition that modulates the rate of sugar absorption and/or metabolism in a subject to whom the composition is administered. The composition includes active soluble fiber, one or more polyphenolic compounds, and a source of amino acids. The composition may be used dry in formulating foodstuffs and beverages. In a preferred embodiment, the composition is a component of a finished beverage, preferably an aqueous beverage, such as a carbonated soft drink.

### CROSS-REFERENCES TO RELATED APPLICATIONS

The present is a non-provisional filing of U.S. Provisional Patent Application No. 60/293,657, which was filed on May 25, 2001. Priority is claimed to the provisional application, which is incorporated herein by reference in its entirety for all purposes.

### BRIEF SUMMARY OF THE INVENTION

The present invention provides a carbohydrate modifying formulation of synergistic ingredients, pertaining to the metabolism of mono and disaccharides. Metabolically, the formulation of the invention slows the absorption of sugars, modifies the release of insulin, and stabilizes blood sugar response. Additionally, the oral ingestion of the formulation of the invention prevents or reduces the formation of dental caries by inhibiting the metabolic capability of dental plaque-forming bacteria to convert sugars into erosive, tooth-decaying acids.

The formulations of the invention provide direct and indirect positive effects on sugar metabolism and blood sugar response. Thus, the formulations of the invention, when consumed in normal amounts, do not adversely contribute or aggravate such conditions as obesity, diabetes, or dietary-based, hormone related hyperactivity such as that often described in young children.

A formulation of the invention may be in liquid or dry form. That is, it may be in the form of a powder that comprises or contains the formulation, or in the form of a liquid, either an aqueous liquid or a non-aqueous liquid. In one preferred aspect the invention provides a finished, water-based beverage, into which the formulation of the invention is incorporated. Moreover, the invention provides a finished water-based beverage, which is acidified and which includes the formulation of the invention.

The invention also includes a method of slowing absorption of sugars, for instance, from the intestine of a subject (including but not limited to a human individual), that comprises administering to the subject, or making available for ingestion by the mammal, a formulation of the invention. The formulation becomes effective when in an aqueous medium, which may be provided extrinsically, for instance by oral or intravenous administration or ingestion of an aqueous liquid containing the formulation, or intrinsically, for instance by ingestion of a solid formulation of the invention which is acted on by the body's digestive secretions and conveyed to and through the body's digestive system (an aqueous environment).

Additional objects and advantages of the invention will be apparent from the detailed description that follows.

### DETAILED DESCRIPTION OF THE INVENTION AND THE PREFERRED EMBODIMENTS

The present invention provides a formulation having desirable properties built upon synergistic ingredients; maintaining low simple sugar levels; and slowing down the normally rapid absorption of simple sugars from the gut. This objective best optimizes energy levels by thwarting the potential destabilizing effects on blood sugar and insulin response, by preferably utilizing a polysaccharide matrix of complex carbohydrates and soluble gum fibers.

The invention provides numerous advantages not found in other agents including, but not limited to, limiting the effects of excessive use of ingredients, such as sugar, that may promote greater oxidative stress and actually reduce energy. Ingredients are preferably chosen from among those that neutralize and inhibit free radical production and oxidative stress and, therefore, help to protect the cellular energy generating mechanisms. Moreover, presently preferred ingredients are those that assist in the cellular utilization and burning of fuels for energy. The composition of the invention also provides multiple tiered use of various timed caloric energy fuels plus the sweetness system disclosed herein for longer, sustained energy.

The present invention provides compositions of active and, optionally, inactive ingredients. The compositions can be prepared in any form including, but not limited to, dry formulations, aqueous formulations, and the like. The compositions of the invention can be included in substantially any manufactured foodstuff or beverage. When consumed simultaneously or in time proximity with other foods that contain sugars, the carbohydrate modifying effects of the compositions will extend to and similarly influence those sugars that are undergoing digestion and assimilation.

A presently preferred embodiment of the composition includes one or more polyphenolic compounds. While not being bound to any particular theory of operation, the inventors presently prefer polyphenolic compounds that inhibit the digestive enzymes amylase (starch digestion) and sucrase (sugar digestion), thereby slowing sugar absorption, and reducing overstimulation of the insulin response, and the subsequent modification of sugar metabolism.

Moreover, preferred polyphenolic compounds inhibit the activity of the bacterial enzyme, glucan transferase, which metabolizes simple sugars as found in beverages, into sticky dental plaque. Without the sticky plaque present, the bacteria cannot adhere to the tooth surfaces, ferment the sugars into acids, and create dental caries.

Polyphenolic compounds of use in the present invention are isolated from any convenient source. Preferred polyphenolic compounds include catechins, tannin extracts, extracts of Camellia Sinensis (e.g., green and black teas), and those found in cranberry, aronia berry, bilberry, and grape seed. Other useful sources of polyphenolic compounds will be apparent to those of skill in the art.

Preferred green tea and black teas actives are the catechins and the aflavins.

The polyphenols can be present in the formulation in any useful amount, but they are preferably present in an amount of from about 0.2 mg to about 500 mg (in 8 oz of water or other diluent, when a diluent is used), preferably from about 10 to about 500 mg.

The formulations of the invention also preferably include one or more amino acid or source of amino acid, preferably selected from soy, soy sprouts or other legume derived proteins such as mung bean, or dairy based protein, amino chelated minerals, and whey or other dairy-based proteins. Other useful sources of amino acid are known to those of skill in the art.

The amino acids of use in the present invention are preferably free amino acids, most preferably free glycine and arginine, which lower blood sugar levels by virtue of mild inducement of insulin release from the pancreas. Arginine, independent of insulin release, also stimulates release of GH (growth hormone) from the pituitary gland. GH is a natural counterbalance to the excessive hypoglycemic effects of insulin. Moreover, glycine, independent of energy dynamics, is an amino acid neurotransmitter substrate, that is described in the scientific literature as being inhibitory to neurological hyperactivity.

Thus, a presently preferred source of amino acid is soy protein, which is a rich source of glycine and arginine, improves glucose tolerance and peripheral insulin sensitivity which is crucial for blood sugar stability.

The one or more amino acid can be present in the composition in any useful amount, but is preferably present in an amount of from about 5 mg to about 10 grams (per 8 ounces of water or other diluent, when a diluent is used), preferably from about 200 mg to about 10 grams. When the amino acid is provided by a source of amino acid, other than the free amino acid, the source is preferably present in an amount that provides the preferred amount of the free amino acid.

Also present in preferred formulations of the invention is soluble fiber, preferably active soluble fiber. As used herein, “active soluble fiber” refers to soluble fiber that is biologically responsive to bacteria in the mammalian GI tract and/or participates in one or more blood sugar modifying mechanism in vivo. The soluble fiber is from any source, however, preferred fibers are those that participate in one or more blood sugar modifying mechanism, such as: conversion of the soluble fiber into short chain fatty acids (SCFAs) by the intestinal bacteria (SCFA, particularly propionic acids, increase glycolysis and reduces gluconeogenesis thus normalizing blood sugar); and (2) slowing of the absorption of sugar from the intestinal tract by the soluble fiber, which ultimately influences the rate of sugar metabolism.

Additionally, antioxidants, including phenolic-based botanical extracts are optionally included as a component of the present formulation. The presence of the antioxidant can aid in overcoming or blunting the pro-oxidant and destabilizing hypoglycemic effects of quickly absorbed simple sugars found in most commercial beverages.

Presently preferred soluble fibers having the above-described characteristics include, inulin, FOS (fructo-oligosaccharides e.g., Beflora™), and gums.

The soluble fiber is present in any useful amount, but is preferably present in an amount of from about 100 mg to about 8 grams (per 8 ounces of water or other diluent, when a diluent is used), preferably from about 500 mg to about 8 grams. In those embodiments in which 5 grams or more soluble fiber is present, the composition of the invention is preferably able to reduce the post prandial rise in blood sugar levels.

In another preferred embodiment of the invention, the formulation includes one or more zinc salt or other source of the zinc ion. Metabolically, zinc is a critical nutrient in the synthesis of insulin and the metabolism of carbohydrates. From a dental perspective, cariogenic bacteria enzymatically produce an insoluble glucan deposit from simple sugars present in the mouth that firmly adheres to the enamel tooth surface. Original study at UCSF School of Preventive Dentistry by the present inventors, demonstrated that two tested zinc salts, 0.5% zinc solution (zinc chloride) and same concentration of zinc ascorbate, both inhibited the growth and adherence of mutans streptococci and sobrinus streptococci in vitro. This demonstrates that the zinc cation, not the counter ion, is the most significant portion of the salt molecule for this function.

Preferred sources of the zinc ion include zinc chloride, zinc sulfate, zinc ascorbate, zinc picolinate, zinc amino acid chelates, and zinc-EDTA.

The zinc salt(s)or source(s) is present in any useful quantity, but is preferably present in an amount of from about 1 mg to about 40 mg (per 8 ounces of water or other diluent, when a diluent is used).

In another preferred embodiment, the invention provides an acidic finished drink composition. The drink is preferably water-based. The water used to formulate the drink can be, for example, still, carbonated, or dairy-based. The pH of the finished drink is preferably from about 1 to about 7, more preferably from about 1 to about 5, and more preferably from about 1 to about 3. The solubility and assimilation of mineral salts, especially divalent minerals such as calcium, zinc, magnesium, iron, are enhanced in an acidic medium. These elements have many important roles relating to cellular metabolism and tissue structure.

The acid or source of acid includes both organic and inorganic acids. Exemplary organic acids include, citric, lactic, tartaric, malic, and ascorbic. Exemplary inorganic acids include phosphoric acid.

The source of the sugars relevant to the operation of the composition of the invention can be contained in the inventive formulation itself, or they may be derived from other foodstuffs.

Also provided by the present invention is a method for modulating sugar metabolism in a mammalian subject. The method includes administering to the subject a composition of the invention, thereby modulating sugar metabolism of the subject. The method also includes making a composition of the invention available to the subject for ingestion, for instance by providing it through retail outlets or through a dispensing physician or other health care provider. The compositions may be provided per se, or may be contained in food supplements or food products. In a preferred embodiment, the moderating results in a decrease of the rate of sugar metabolism relative to the rate in the absence of a composition of the invention. In another preferred embodiment, the moderating includes a linearization of the rate of metabolism, eliminating spikes and/or valleys in the sugar metabolism profile, and/or decreasing the peak height and/or valley depth in the sugar metabolism profile. In another preferred embodiment, the sugar metabolism is modulating by the composition effecting a decrease in the absorption rate of the sugar by the mammalian gut.

It should be noted that the compositions of this invention are not sweeteners, sugar substitutes, or flavor enhancers. To the contrary, by themselves they tend to be bland and to offer no significant flavor notes to a formula that contains them.

The following examples are provided by way of illustration only and not by way of limitation. Those of skill in the art will readily recognize a variety of non-critical parameters that could be changed or modified to yield essentially similar results.

### EXAMPLES

### Example 1

Yogurt Drink (8 oz):

Combine yogurt cultured milk, fresh fruit and sugar (10 g) or intense sweetener of choice (10 ppm) with the modifying agent. The modifying agent includes inulin fiber (3 g), bilberry, citrus bioflavonoids, green tea extract mix (polyphenolic) (100 mg); soy isoflavones (50 mg); zinc sulfate (7 mg); and vitamin C (60 mg). Add sufficient water to bring volume to 8 ounces.

### Example 2

Soft Drink (8 oz):

Combine water (still or carbonated), flavor (natural or artificial) and sugar (10 gm) or an intense sweetener of choice (10 ppm) with the modifying agent.

The modifying agents includes inulin fiber (3 g), bilberry, citrus bioflavonoids, green tea extract mix (polyphenolic) (100 mg); soy isoflavones (50 mg); zinc sulfate (7 mg); and vitamin C (60 mg); soy protein extract (50 mg); and mineral amino chelates (amino acid glycine) (300 mg).

The present invention provides a novel beverage that includes components that have sugar metabolism regulating properties. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

What is claimed is:

1. An aqueous formulation comprising:
(a) a polyphenolic compound;
(b) an amino acid;
(c) active soluble fiber; and
(d) an aqueous diluent.

2. The formulation according to claim 1, wherein said polyphenolic compound is a member selected from the group consisting of catechins, aflavins and combinations thereof.

3. The formulation according to claim 2, wherein said polyphenolic compound inhibits a mammalian digestive enzyme.

4. The formulation according to claim 3, wherein said digestive enzyme is a member selected from the group consisting of amylase, sucrase and combinations thereof.

5. The formulation according to claim 3, wherein said polyphenolic compound is present in an amount sufficient to inhibit said enzyme.

6. The formulation according to claim 1, in which said polyphenolic compound, when administered to a mammalian subject, has a property which is a member of the group consisting of slowing sugar absorption in said subject, reducing overstimulation of insulin response in said subject and combinations thereof.

7. The formulation according to claim 6, wherein said polyphenolic compound is present in an amount sufficient to reduce in a mammalian subject a member selected from the group consisting of sugar absorption, overstimulation of immune response and combinations thereof.

8. The formulation according to claim 1, wherein said polyphenolic compound inhibits a bacterial enzyme.

9. The formulation according to claim 8, wherein said polyphenolic compound is present in an amount sufficient to inhibit said bacterial enzyme in a mammalian subject.

10. The formulation according to claim 8, wherein said bacterial enzyme is glucan transferase.

11. The formulation according to claim 10, having an inhibitory effect on formation of dental caries.

12. The formulation according to claim 1, wherein said polyphenolic is derived from a member selected from the group consisting of tannin, extracts of Camellia sinensis, bilberry, grapeseed and combinations thereof.

13. The formulation according to claim 1, wherein said polyphenolic is present in said formulation in an amount of from about 0.2 mg to about 500 mg in about 8 ounces of said formulation.

14. The composition according to claim 13, wherein said polyphenolic is present in said formulation in an amount of from about 1 mg to about 200 mg in about 8 ounces of said formulation.

15. The formulation according to claim 1, wherein said amino acid is present in a source of amino acid which is a member selected from the group consisting of soy protein extract, an amino chelated mineral, whey protein and combinations thereof.

16. The formulation according to claim 1, wherein said amino acid lowers blood sugar levels in a subject.

17. The formulation according to claim 16, wherein said amino acid is present in an amount sufficient to lower said blood sugar level in said subject.

18. The formulation according to claim 16, wherein said amino acid lowers said blood sugar by inducing insulin release from the pancreas of said subject.

19. The formulation according to claim 16, wherein said amino acid lowers said blood sugar in said subject by inducing growth hormone release from the pituitary gland of said subject.

20. The formulation according to claim 16, wherein said amino acid is present in said formulation in an amount of from about 1 mg to about 50 mg in about 8 ounces of said formulation.

21. The formulation according to claim 20, wherein said amino acid is present in an amount of from about 3 mg to about 10 mg in about 8 ounces of said formulation.

22. The formulation according to claim 21, wherein said amino acid is present in an amount of from about 5 mg to about 7 mg in about 8 ounces of said formulation.

23. The formulation according to claim 1, wherein said soluble fiber is a member selected from the group consisting of inulin, fructo-oligosaccharides and gums.

24. The formulation according to claim 1, wherein said soluble fiber modifies blood sugar level in a subject.

25. The formulation according to claim 24, wherein said soluble fiber is present in said formulation in an amount sufficient to lower said blood sugar level.

26. The formulation according to claim 24, wherein said blood sugar level is modified by a mechanism that is a member selected from the group consisting of conversion of said soluble fiber into short chain fatty acids, slowing absorption of sugar from the intestinal tract, and combinations thereof.

27. The formulation according to claim 24, wherein said soluble fiber is present in an amount of from about 100 mg to about 8 grams in about 8 ounces of said formulation.

28. The formulation according to claim 24, wherein said soluble fiber is present in an amount of at least about 3 grams in about 8 ounces of said formulation.

29. The formulation according to claim 1, further comprising a metal ion.

30. The formulation according to claim 29, wherein said metal ion is Zn⁺².

31. The formulation according to claim 29, wherein said metal ion is present in said formulation in an amount sufficient to inhibit growth and adherence in a subject of a member selected from the group consisting of mutans streptococci, sobrinus streptococci and combinations thereof.

32. The formulation according to claim 31, wherein said metal ion is present in said formulation in an amount of from about 1 mg to about 40 mg in about 8 ounces of said formulation.

33. The formulation according to claim 1, wherein said formulation is acid finished.

34. The formulation according to claim 33, wherein said acid is a member selected from the group consisting of citric acid, lactic acid, tartaric acid, malic acid, ascorbic acid, phosphoric acid and combinations thereof.

35. A formulation that slows the absorption of sugars from mammalian intestine, said formulation comprising the elements:
(a) a polyphenolic compound;
(b) an amino acid;
(c) active soluble fiber; and
(d) an aqueous diluent,
wherein each element is present in an amount sufficient to provide said formulation that slows said absorption of sugars.

36. A formulation according to claim 35, which retards the formation of dental caries, said formulation further comprising a metal ion in an amount sufficient to retard said formation of said dental caries.

37. A composition comprising:
(a) a polyphenolic compound;
(b) an amino acid; and
(c) active soluble fiber.

38. A formulation that slows the absorption of sugars from mammalian intestine, said formulation comprising the elements:
(a) a polyphenolic compound;
(b) an amino acid; and
(c) active soluble fiber,
wherein each element is present in an amount sufficient to provide said formulation that slows said absorption of sugars.

39. A method of modulating sugar metabolism in a mammalian subject, said method comprising administering to said subject, making available for ingestion by said subject, or ingesting by said subject, an amount of the formulation according to claim 1, effective to modulate said sugar metabolism.
